(19) United States
(12) Patent Application Publication
(10) Pub. No.: US 2023/0364148 A1
(43) Pub. Date: Nov. 16, 2023

(54) INHIBITION AND REVERSION OF CHRONIC OBSTRUCTIVE PULMONARY DISEASE (COPD) BY ENDOTHELIAL CELL REGENERATION

(71) Applicant: Therapeutic Solutions International, Inc., Oceanside, CA (US)

(72) Inventors: Thomas E. ICHIM, Oceanside, CA (US); Timothy G. DIXON, Oceanside, CA (US); Zeeshan PIRACHA, Oceanside, CA (US); James VELTMEYER, Oceanside, CA (US)

(73) Assignee: Therapeutic Solutions International, Inc., Oceanside, CA (US)

(21) Appl. No.: 18/316,084

(22) Filed: May 11, 2023

Related U.S. Application Data
(60) Provisional application No. 63/341,064, filed on May 12, 2022.

(51) Int. Cl. A61K 35/28; A61K 35/44; A61K 38/19; A61P 11/00

(52) U.S. Cl. CPC A61K 35/28; A61K 35/44; A61K 38/193; A61P 11/00

(57) ABSTRACT

Disclosed are means, treatment methods, and compositions of matter useful for prevention and/or reversion of chronic obstructive pulmonary disease (COPD). In one embodiment the invention provides the administration of mesenchymal stem cells and exosome thereof as a means of augmenting endogenous endothelial regeneration and/or endothelial regeneration stimulated by exogenous means. In some embodiments the invention provides administration of allogeneic mesenchymal stem cells together with autologous endothelial progenitor cells and/or mobilization of said autologous endothelial progenitor cells.

### CROSS-REFERENCE TO RELATED APPLICATIONS

This application claims priority to U.S. Provisional Application No. 63/341,064, titled “Inhibition and Reversion of Chronic Obstructive Pulmonary Disease (COPD) by Endothelial Cell Regeneration” filed May 12, 2022, which is hereby incorporated by reference herein in its entirety.

### FIELD OF THE INVENTION

The invention relates to the use of regenerative stem cell populations for treating chronic obstructive pulmonary disease.

### BACKGROUND OF THE INVENTION

COPD is a consistently progressive, ultimately fatal disease for which no treatment exists capable of either reversing or even interrupting its course. It afflicts more than 5% of the population in many countries, and it accordingly represents the third most frequent cause of death in the U.S., where it accounts for more than 600 billion in health care costs, morbidity, and mortality.

COPD possesses several features making it ideal for stem cell-based interventions: a) the quality of life and lack of progress demands the ethical exploration of novel approaches. For example, bone marrow stem cells have been used in over a thousand cardiac patients with some indication of efficacy [1, 2].

Mesenchymal Stem Cells (MSCs) are potent immunomodulatory cells that recognize sites of injury, limit effector T cell reactions, and stimulate regulatory cell populations (i.e., T-regs) via growth factors, cytokines, and other mediators. Simultaneously, MSCs also stimulate local tissue regeneration via paracrine effects inducing angiogenic, anti-fibrotic and remodeling responses [3]. Consequently, MSCs-based therapy represents a viable treatment option for autoimmune conditions and other inflammatory disorders [4-9], yielding beneficial effects in models of autoimmune Type 1 Diabetes [10-16], Systemic Lupus Erythematosus, Autoimmune Encephalomyelitis [17], Multiple Sclerosis [18, 19], cardiac insufficiency [20, 21], and organ transplantation [22]. MSCs have been reported to inhibit inflammation and fibrosis in the lungs [23-26], have shown safety in clinical trials for ARDS[27-30], and have been recently suggested as useful to treat patients with severe COVID-19 based on their effects preventing or attenuating the immunopathogenic cytokine storm [31-34].

Unfortunately, evaluation of stem cell therapy in COPD has lagged behind other areas of regenerative investigation; b) the underlying cause of COPD appears to be inflammatory and/or immunologically mediated. The destruction of alveolar tissue is associated with T cell reactivity [35, 36], pathological pulmonary macrophage activation [37], and auto-antibody production [38]. Mesenchymal stem cells have been demonstrated to potently suppress autoreactive T cells [39, 40], inhibit macrophage activation [41], and autoantibody responses [12]. Additionally, mesenchymal stem cells can be purified in high concentrations from adipose stromal vascular tissue together with high concentrations of T regulatory cells [42], which in animal models are approximately 100 more potent than peripheral T cells at secreting cytokines therapeutic for COPD such as IL-10 [43, 44]. Additionally, use of adipose derived cells has yielded promising clinical results in autoimmune conditions such as multiple sclerosis [42]; and c) Pulmonary stem cells capable of regenerating damaged parenchymal tissue have been reported [45]. Administration of mesenchymal stem cells into neonatal oxygen-damaged lungs, which results in COPD-like alveoli dysplasia, has been demonstrated to yield improvements in two recent publications [46, 47].

Unfortunately, to date, despite promising “efficacy signals”, no cell based therapy has successfully completed clinical trial requirements for marketing registration.

### SUMMARY

Preferred embodiments are directed to methods of treating Chronic Obstructive Pulmonary Disease (COPD) comprising the steps of: a) obtaining a mesenchymal stem cell population; and b) inducing increasing endothelial progenitor cell numbers and/or activity in the blood.

Preferred methods include embodiments wherein administration of said mesenchymal stem cell population is performed before, concurrently with, or subsequent to increasing endothelial progenitor cell population and/or activity in the blood.

Preferred methods include embodiments wherein said mesenchymal stem cell population is either autologous, allogeneic or xenogeneic.

Preferred methods include embodiments wherein inducing increase in said endothelial progenitor cell population is induced by administration of exogenous endothelial progenitor cells.

Preferred methods include embodiments wherein said exogenous endothelial progenitor cells are derived from a source that is either autologous, allogeneic, or xenogeneic.

Preferred methods include embodiments wherein said exogenous endothelial progenitor cells are obtained from adipose tissue.

Preferred methods include embodiments wherein said exogenous endothelial progenitor cells are obtained from bone marrow.

Preferred methods include embodiments wherein said exogenous endothelial progenitor cells are obtained from umbilical cord tissue.

Preferred embodiments are directed to methods of treating COPD comprising administration of mesenchymal stem cells and myeloid derived suppressor cells.

Preferred methods include embodiments wherein said mesenchymal stem cells are JadiCells.

Preferred embodiments are directed to methods of treating COPD comprising administration of one or more compounds capable of inducing an increase in number and/or activity of myeloid derived suppressor cells combined with a mesenchymal stem cell.

Preferred methods include embodiments wherein said compound capable of increasing number and/or activity of said mesenchymal stem cell is GM-CSF.

Preferred methods include embodiments wherein said mesenchymal stem cell is JadiCell.

Preferred embodiments are directed to methods of treating COPD comprising administration of GM-CSF and JadiCells®.

Preferred embodiments are directed to methods comprising administration of myeloid derived suppressor cells and JadiCells®.

### BRIEF DESCRIPTION OF THE DRAWINGS

FIG. 1 is a bar graph showing polymorphonuclear lymphocyte infiltrations was significantly reduced in mice receiving the combination of JadiCells and Myeloid Derived Suppressor cells.

FIG. 2 is a bar graph showing polymorphonuclear lymphocyte infiltrations was significantly reduced in mice receiving the combination of JadiCells and GM-CSF.

### DETAILED DESCRIPTION OF THE INVENTION

The invention teaches the previously unknown and unexpected findings that mesenchymal stem cell prophylactic and/or therapeutic activity for COPD is substantially augmented by specific immunological cells termed “myeloid derived suppressor cells”. In a specific embodiment the invention teaches synergies between GM-CSF and mesenchymal stem cells for reducing COPD pathology.

The first type of myeloid derived suppressor cells is the monocytic myeloid-derived suppressor cells (M-MDSC) and the second type is polymorphonuclear myeloid-derived suppressor cells (PMN-MDSC). About 20-30% of MDSC consists of monocytic cells, i.e., M-MDSC, and are generally associated with high activity of Arginase-1 and iNOS.sup.10. Two different phenotypes (CD11b.sup.+CD14.sup.-CD15.sup.- and CD33.sup.+or CD11b.sup.+CD14.sup.+CD33.sup.+ and HLA-DR.sup.lo) are used to characterize these M-MDSC cells depending on the type of cancer. The second population, i.e., PMN-MDSC, are comprised of granulocytic cells and are usually associated with high level of ROS production.sup.36. PMN-MDSC represent the major population of MDSC (about 60-80%) and represent the most abundant population of MDSC in most types of cancer. PMN-MDSC are phenotypically and morphologically similar to neutrophils (PMN) and share the CD11b+CD14-CD15+/CD66b+phenotype. The may also be characterized as CD33.sup.+. PMN-MDSC are important regulators of immune responses in cancer and have been directly implicated in promotion of tumor progression. However, the heterogeneity of these cells and lack of distinct markers hampers the progress in understanding of the biology and clinical significance of these cells. One of the major obstacles in the identification of PMN-MDSC is that they share the same phenotype with normal polymorphonuclear cells (PMN).

The administration of myeloid derived suppressor cells may be performed by using the cells themselves or by pre-activating them. In certain embodiments of the invention, small molecules of the invention are used to sustain and enhance the immune suppressive functions of MDSCs by preventing the MDSCs to undergo maturation and terminal differentiation. Through this process the growth factor producing properties of the myeloid suppressor cells are retained and/or enhanced.

For the purpose of the invention, we describe the immature stage of MDSCs as being characterized by low cell surface expression of MHC class II, co-stimulatory molecules, e.g., CD80, CD86, CD40, low CD11c and F4/80. Immature MDSCs arc further characterized by a large nucleus to cytoplasm ratio and an immunosuppressive activity. In some cases enhancement of growth factor properties is produced by treatment of the cells by histone deacetylase inhibitors such as decitabine. In some embodiments of the invention, MDSCs are autologously-derived cells. For example, MDSCs may be isolated from normal adult bone marrow or from sites of normal hematopoiesis, such as the spleen. Obviously, splenic sources of MDSC are difficult in clinical situations. MDSCs are scant in the periphery and are present in a low number in the bone marrow of healthy individuals. However, they are accumulated in the periphery when intense hematopoiesis occurs. Upon distress due to graft-versus-host disease (GVHD), cyclophosphamide injection, or g-irradiation, for example, MDSCs may be found in the adult spleen. Thus, in certain embodiments, MDSCs may be isolated from the adult spleen. MDSCs may also be isolated from the bone marrow and spleens of tumor-bearing or newborn mice. In a preferred embodiment, MDSCs are isolated in vivo by mobilizing MDSCs from hematopoietic stem cells (HSCs) or bone marrow suing stem cell mobilizers such as G-CSF Any suitable stem cell mobilizer or combination of mobilizers is contemplated for use in the present invention. MDSCs may be induced endogenously and/or be collected from the blood e.g., by apheresis, following treatment of a subject or patient with the stem cell mobilizer(s). In certain embodiments, MDSCs can be derived, for example, in vitro from a patient's HSCs, from MHC matching ES cells, induced pluripotent stem (iPS) cells Specifically, isolated hematopoietic stem cells (HSCs) can be stimulated to differentiate into Gr-1+/CD11b+, Gr-1+/CD11b.+/CD115+, Gr-1+/CD11b+/F4/80+, or Gr-1+/CD11b+/CD115+/F4/80+MDSCs by culturing in the presence of stem-cell factor (SCF) or SCF with tumor factors, which can increase the MDSC population. The culture conditions for mouse and human HSCs are described in detail in U.S. Publication No. 2008/0305079 by Chen. In further embodiments, other cytokines may be used, e.g., VEGF, GM-CSF, M-CSF, SCF, S100A9, TPO, IL-6, IL-1, PGE-2 or G-CSF to stimulate MDSC differentiation from HSCs in vitro. Any one of the cytokines may be used alone or in combination with other cytokines. In still another embodiment, tumor-conditioned media may be used with or without SCF to stimulate HSCs to differentiate into MDSCs. In other embodiments, MDSCs are allogeneic cells, such as MDSCs obtained or isolated from a donor or cell line. MDSC cell lines and exemplary methods for their generation are well known in the art and are described in the literature.

The invention provides administration of myeloid derived suppressor cells, and/or exosomes derived from such cells as a treatment for copd degeneration. One of ordinary skill in the art may readily determine the appropriate concentration, or dose of the myeloid derived suppressor cells disclosed herein for therapeutic administration. The ordinary artisan will recognize that a preferred dose is one that produces a therapeutic effect, such as preventing, treating and/or reducing inflammation associated with copd diseases, disorders and injuries, in a patient in need thereof. Of course, proper doses of the cells will require empirical determination at time of use based on several variables including but not limited to the severity and type of disease, injury, disorder or condition being treated; patient age, weight, sex, health; other medications and treatments being administered to the patient; and the like. An exemplary dose is in the range of about 0.25-2.0 .times.10.sup.6 cells. Other dose ranges include 0.1-10.0 .times.10.sup.6,7,8,9,10,11, or 10.sup.12 cells per dose or injection regimen. An effective amount of cells may be administered in one dose, but is not restricted to one dose. Thus, the administration can be two, three, four, five, six, seven, eight, nine, ten, eleven, twelve, thirteen, fourteen, fifteen, sixteen, seventeen, eighteen, nineteen, twenty, or more, administrations of pharmaceutical composition. Where there is more than one administration of a pharmaceutical composition in the present methods, the administrations can be spaced by time intervals of one minute, two minutes, three, four, five, six, seven, eight, nine, ten, or more minutes, by intervals of about one hour, two hours, three, four, five, six, seven, eight, nine, ten, 11, 12, 13, 14, 15, 16, 17, 18, 19, 20, 21, 22, 23, 24 hours, and so on. In the context of hours, the term “about” means plus or minus any time interval within 30 minutes. The administrations can also be spaced by time intervals of one day, two days, three days, four days, five days, six days, seven days, eight days, nine days, ten days, 11 days, 12 days, 13 days, 14 days, 15 days, 16 days, 17 days, 18 days, 19 days, 20 days, 21 days, and combinations thereof. The invention is not limited to dosing intervals that are spaced equally in time, but encompass doses at non-equal intervals. A dosing schedule of, for example, once/week, twice/week, three times/week, four times/week, five times/week, six times/week, seven times/week, once every two weeks, once every three weeks, once every four weeks, once every five weeks, and the like, is available for the invention. The dosing schedules encompass dosing for a total period of time of, for example, one week, two weeks, three weeks, four weeks, five weeks, six weeks, two months, three months, four months, five months, six months, seven months, eight months, nine months, ten months, eleven months, and twelve months. Provided are cycles of the above dosing schedules. The cycle can be repeated about, e.g., every seven days; every 14 days; every 21 days; every 28 days; every 35 days; 42 days; every 49 days; every 56 days; every 63 days; every 70 days; and the like. An interval of non-dosing can occur between a cycle, where the interval can be about, e.g., seven days; 14 days; 21 days; 28 days; 35 days; 42 days; 49 days; 56 days; 63 days; 70 days; and the like. In this context, the term “about” means plus or minus one day, plus or minus two days, plus or minus three days, plus or minus four days, plus or minus five days, plus or minus six days, or plus or minus seven days. Cells derived from the methods of the present invention may be formulated for administration according to any of the methods disclosed herein in any conventional manner using one or more physiologically acceptable carriers optionally comprising excipients and auxiliaries. Proper formulation is dependent upon the route of administration chosen. The compositions may also be administered to the individual in one or more physiologically acceptable carriers. Carriers for cells may include, but are not limited to, solutions of normal saline, phosphate buffered saline (PBS), lactated Ringer's solution containing a mixture of salts in physiologic concentrations, or cell culture medium. In further embodiments of the present invention, at least one additional agent may be combined with the copd-derived progenitor cells of the present invention for administration to an individual according to any of the methods disclosed herein. Such agents may act synergistically with the cells of the invention to enhance the therapeutic effect. Such agents include, but are not limited to, growth factors, cytokines, chemokines, antibodies, inhibitors, antibiotics, immunosuppressive agents, steroids, anti-fungals, anti-virals or other cell types (i.e. stem cells or stem-like cells, for example AMP cells), extracellular matrix components such as aggrecan, versican hyaluronic acid and other glycosaminoglycans, collagens, etc. Inactive agents include carriers, diluents, stabilizers, gelling agents, delivery vehicles, ECMs (natural and synthetic), scaffolds, and the like. When the cells of the present invention are administered conjointly with other pharmaceutically active agents, even less of the cells may be needed to be therapeutically effective. The timing of administration of myeloid derived suppressor cells 1-based compositions will depend upon the type and severity of the copd disease, disorder, or injury being treated. In one embodiment, the cell-based compositions are administered as soon as possible after onset of symptoms, diagnosis or injury. In another embodiment, cell-based compositions are administered more than one time following onset of symptoms, diagnosis or injury. In certain embodiments, where surgery is required, the cell-based compositions are administered at surgery. In still other embodiments, the cell-based compositions are administered at as well as after surgery. Such post-surgical administration may take the form of a single administration or multiple administrations.

In some embodiments, the myeloid derived suppressor cells are administered parenterally to the individual. The terms “parenteral administration” and “administered parenterally” are art-recognized and refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articulare, subcapsular, subarachnoid, intraspinal, epidural, intracerebral, intraosseous, intracopdnous, and intrasternal injection or infusion. Support matrices, scaffolds, membranes and the like into which the cell-based compositions can be incorporated or embedded include matrices which are recipient-compatible and which degrade into products which are not harmful to the recipient. Detailed information on suitable support matrices, etc. can be found in U.S. Pat. Nos. 8,058,066 and 8,088,732, both of which are incorporated herein by reference.

In certain aspects of the invention, the small compound glatiramer acetate (GA) (Copolymer 1/Copaxone) is used to modify MDSC function. In another aspect, a small compound MAP kinase inhibitor is used to modify MDSC function. In yet another aspect, GA and a small compound MAP kinase inhibitor, such as, e.g., a c-Jun N-terminal kinase (JNK) small compound inhibitor, have a surprising synergistic effect on the modulation of MDSC function for the treatment or prevention of alloimmune response and pro-inflammatory immune responses.

In some embodiments MDSC are pre-activated before their administration into tissue possessing degenerated copd. We describe the use of small molecules to regulate biological signals in order to alter the properties of MDSC. Signal regulation by small compounds (e.g., small molecule inhibitors) can control cell differentiation and function in a controllable and reproducible manner according to the current invention.

The term “small compound” as used herein refers to compounds, chemicals, small molecules, small molecule inhibitors, or other factors that are useful for modulating MDSC function. Small molecule inhibitors have been used as immunosuppressive and anti-inflammatory drugs. GA (Copolymer 1/Copaxone) is an FDA approved drug for the treatment of multiple sclerosis, a T cell-mediated autoimmune disease. SP600125 is a small compound inhibitor of JNK, which is a downstream molecule of a number of signaling pathways that regulate both innate and adaptive immunity. The present invention is related to the discovery that these small compounds can regulate the suppressive functions of MDSCs to facilitate the establishment of immune tolerance. In one embodiment of the invention GA is administered systemically as a treatment of copd degeneration. In another embodiment treatment of osteoarthritis by GA is disclosed. It has been known for a while that GA alone has not been effective for treating autoimmune diseases. Specifically, GA is known to be only partially effective for treating the autoimmune disease multiple sclerosis [Johnson et al. (1995) Neurology 45: 1268-1276]. Moreover, clinical studies using GA for the treatment of IBD were discontinued, because GA failed to treat IBD. The present invention is based on the discovery that administration of GA in combination with MDSCs, or with MDSCs and a MAP kinase inhibitor (e.g., SP600125), is surprisingly effective for the treatment of the autoimmune disease, IBD. It is presently discovered that GA and SP600125 have a synergistic effect in combination. In order to increase therapeutic efficacy in some cases, GA is administered intra-articularly and/or by depot or drug delivery mechanisms in order to enhance the concentration locally without inducing systemic effects. In other embodiments MDSC are first-pretreated before administration of cells intra-articularly. In some embodiments GA is administered together with autologous bone marrow cells.

For practical implementation of the invention, in some embodiments autologous non-expanded cells are provided to a patient with copd degeneration while the patient is concurrently receiving the treatment COPAXONE™ which is the brand name for GA (formerly known as copolymer-1). GA, the active ingredient of COPAXONE™, is a random polymer consisting of the acetate salts of synthetic polypeptides, containing four naturally occurring amino acids: L-glutamic acid, L-alanine, L-tyrosine, and L-lysine with an average molar fraction of 0.141, 0.427, 0.095, and 0.338, respectively [CAS number 147245-92-9]. The average molecular weight of GA is 4,700 11,000 daltons. Chemically, glatiramer acetate is designated L-glutamic acid polymer with L-alanine, L-lysine and L-tyrosine, acetate (salt). GA is a random polymer composed of tyrosine, glutamic acid, alanine and lysine, that has been used for the treatment of multiple sclerosis, a T cell-mediated autoimmune disease. GA may be obtained from Teva Pharmaceutical Industries (Petach Tikva, Israel). For the practice of the present invention, variants, modified forms and/or derivatives of GA are also contemplated for use in the present invention. One of skill in the art can readily substitute structurally-related amino acids for GA without deviating from the spirit of the invention. The present invention includes polypeptides and peptides which contain amino acids that are structurally related to tyrosine, glutamic acid, alanine or lysine and possess the ability to stimulate polyclonal antibody production upon introduction. Such substitutions retain substantially equivalent biological activity in their ability to suppress autoimmune diseases such as IBD, and alloimmune responses, such as GVHD and organ transplantation rejection. These substitutions are structurally-related amino acid substitutions, including those amino acids which have about the same charge, hydrophobicity and size as tyrosine, glutamic acid, alanine or lysine. For example lysine is structurally-related to arginine and histidine; glutamic acid is structurally-related to aspartic acid; tyrosine is structurally-related to serine, threonine, phenylalanine and tryptophan; and alanine is structurally-related to valine, leucine and isoleucine. These and other conservative substitutions, such as structurally-related synthetic amino acids, are contemplated by the present invention. Any one or more of the amino acids in GA may be substituted with l- or d-amino acids. As is known by one of skill in the art, l-amino acids occur in most natural proteins. However, d-amino acids are commercially available and can be substituted for some or all of the amino acids used to make GA. Thus, in some embodiments, the present invention contemplates GA formed from mixtures of d- and l-amino acids.

In certain aspects, the present invention provides compositions comprising MDSCs and small compounds. For example, compositions comprising MDSCs in combination with GA and/or a MAP kinase inhibitor are provided. In a preferred embodiment, MDSCs are administered with GA and a MAP kinase inhibitor. In some aspects, MDSCs are derived from bone marrow or HSCs in vitro. In another aspect, MDSCs are freshly isolated from a patient or donor, as described, supra. The MDSCs of the invention may be autologous or allogeneic. In yet other aspects of the invention, a subject or patient is administered a composition containing MDSCs and one or more small compounds of the invention. Administration may be achieved by any suitable method. In yet another aspect, a subject is administered MDSCs and one or more small compounds of the invention, each as a separate composition. For example, a subject may be administered one composition containing MDSCs and one or more compositions each containing one or more small compound, such as, e.g., GA and/or SP600125. Such compositions may be administered to at the same or different times via the same or different routes of administration.

There are several therapeutic embodiments that are useful for the education of a practitioner of the invention. In one embodiment of the invention, a patient is administered a composition containing at least one stem cell mobilizer, such as, but not limited to G-CSF, AMD 3100, CTCE-9908, FTY720, Flt3 ligand, SCF, S100A9, GM-CSF and M-CSF. These agents would increase the amount of MDSC into circulation

The patient is further administered one or more additional compositions containing one or more small compounds of the invention for enhancing the suppressive activity of MDSCs, such as GA and/or SP600125. In certain aspects of the invention, these compositions may be administered at the same or different times and at the same or different sites. In another aspect, stem cell mobilizing agents and small compounds of the invention may be administered as a single composition. The compositions of the invention can be formulated for administration in any convenient way for use in human or veterinary medicine. The MDSCs of the invention may be incorporated into liposomes, microemulsions, micelles, unilamellar or multilamellar vesicles, erythrocyte ghosts or spheroblasts. In one embodiment, the MDSCs, stem cell mobilizing agents and/or small compounds of the invention can be delivered in one or more vesicles, including as a liposome (see Langer, Science, 1990; 249:1527-1533; Treat et al., in Liposomes in the Therapy of Infectious Disease and Cancer, Lopez-Berestein and Fidler (eds.), Liss: New York, pp. 353-365 (1989); Lopez-Berestein, ibid., pp. 317-327; see generally ibid.).

For the practice of the invention, in yet another embodiment, MDSCs and small compounds of the invention can be delivered in a controlled release form. In some example decellularized placental tissue is utilized as a delivery mechanism. There are other means of deliver that may be utilized, for example, one or more small compounds (e.g., GA and/or SP600125) may be administered in a polymer matrix such as poly (lactide-co-glycolide) (PLGA), in a microsphere or liposome implanted subcutaneously, or by another mode of delivery (see, Cao et al., 1999, Biomaterials, February; 20(4):329-39). Another aspect of delivery includes the suspension of the compositions of the invention in an alginate hydrogel. Additionally the use of micropumps is also disclosed.

When we speak of “therapeutically effective” we are referring to a dose or an amount refers to that quantity of a compound or pharmaceutical composition that is sufficient to result in a desired activity upon administration to a mammal in need thereof. As used herein, the term “therapeutically effective amount/dose” refers to the amount/dose of a pharmaceutical composition of the invention that is suitable for treating a patient or subject having an autoimmune disease. In certain embodiments of the invention the patient or subject may be a mammal. In certain embodiments, the mammal may be a human.

The present invention also provides pharmaceutical formulations or dosage forms for administration to mammals in need thereof. The subject invention also concerns the use of GA or a GA derivative and/or MAP kinase inhibitors, such as, e.g., SP600125, in the preparation of a pharmaceutical composition. In some embodiments, a pharmaceutical composition of the invention includes MDSCs and GA and/or a small compound inhibitor of a MAP kinase. In a specific embodiment, the inhibitor is a small compound inhibitor of JNK. In yet another embodiment, the pharmaceutical composition includes MDSCs, GA and a small compound MAP kinase inhibitor. The pharmaceutical compositions of the invention optionally include a pharmaceutically acceptable carrier or diluent.

The compositions and formulations of the present invention can be administered topically, parenterally, orally, by inhalation, as a suppository, or by other methods known in the art. The term “parenteral” includes injection (for example, intravenous, intraperitoneal, epidural, intrathecal, intramuscular, intraluminal, intratracheal or subcutaneous). The preferred routes of administration are intravenous (i.v.), intraperitoneal (i.p.) and subcutaneous (s.c.) injection. When MDSCs are administered separately from the small compounds of the invention, the preferred route of administration is i.v. However, MDSCs may also be administered subcutaneously or intraperitoneally. The preferred route of administration for GA and the stem cell mobilizers of the invention is subcutaneous administration. The preferred route of administration for SP600125 is i.p. injection. However, the stem cell mobilizers and small compounds of the invention may be administered in any convenient way, including for i.v., s.c., oral, or i.p. injection. Administration of the compositions of the invention may be once a day, twice a day, or more often, but frequency may be decreased during a maintenance phase of the disease or disorder, e.g., once every second or third day instead of every day or twice a day. The dose and the administration frequency will depend on the clinical signs, which confirm maintenance of the remission phase, with the reduction or absence of at least one or more preferably more than one clinical signs of the acute phase known to the person skilled in the art. More generally, dose and frequency will depend in part on recession of pathological signs and clinical and subclinical symptoms of a disease condition or disorder contemplated for treatment with the present compounds.

During the practice of the invention, it will be appreciated that the amount of MDSCs and small compounds of the invention required for use in treatment will vary with the route of administration, the nature of the condition for which treatment is required, and the age, body weight and condition of the patient, and will be ultimately at the discretion of the attendant physician or veterinarian. These compositions will typically contain an effective amount of the compositions of the invention, alone or in combination with an effective amount of any other active material, e.g., those described above. Preliminary doses can be determined according to animal tests, and the scaling of dosages for human administration can be performed according to art-accepted practices.] Keeping the above description in mind, typical dosages of MDSCs for administration to humans range from about 5 .times.10.sup.5 to about 5 .times.10.sup.6 or higher, although lower or higher numbers of MDSCs are also possible. In embodiments in which autologous MDSCs are administered, an advantage of the present invention is that there is little to no toxicity, since the MDSCs are autologous. In a preferred embodiment, a patient may receive, for example, 5 .times.10.sup.7-5 .times.10.sup.10 MDSCs. Keeping the above description in mind, typical dosages of GA for administration to humans may range from about 50 .mu·g/kg (of body weight) to about 50 mg/kg per day. A preferred dose range is on the order of about 100 .mu·g/kg/day to about 10 mg/kg/day, more preferably a range of about 300 .mu·g/kg/day to about 1 mg/kg/day, and still more preferably from about 300 .mu·g/kg/day to about 700 .mu·g/kg/day. The length of treatment, i.e., number of days, will be readily determined by a physician treating the patient, however the number of days of treatment may range from 1 day to about 20 days. In a preferred embodiment, the dose of GA is administered at a frequency of about once every 7 days to about once every day. In a more preferred embodiment, the dose of GA is administered at a frequency of about once every day. Preferably, the number of days of treatment is from about 5 to about 15 days and most preferably from about 10 to about 12 days. In a specific embodiment, a patient may receive, for example, 500 .mu·g/kg/day subcutaneously (SC) for 12 days. In another embodiment of the invention, the dose of GA is administered at a frequency of about once every 30 days to about once every day. In a specific embodiment, GA is administered subcutaneously for 12 days. [See, Weber, M. S., et al., (2007) Nat. Med.; 13(8):935-943.] Keeping the above description in mind, typical dosages of SP600125 for administration to humans range from 50 .mu·g/kg (of body weight) to about 500 mg/kg per day. A preferred dose is about 50 mg/kg/day. Keeping the above description in mind, typical dosages of the stem cell mobilizer Flt3 ligand may range from about 10 .mu·g/kg to about 1000 .mu·g/kg. A preferred dose range is on the order of about 20 .mu·g/kg to about 300 .mu·g/kg. In certain embodiments, a patient may receive, for example, 20 .mu·g/kg of Flt3L per day subcutaneously for 14 days each month [see Disis, M L et al. (2002) Blood. 99: 2845-2850]. Preferably, the length of treatment is at least 5 days. Keeping the above description in mind, typical dosages of G-CSF may range from about 2 to about 12 mg/kg/day. The length of treatment may range from about 1 day to about 14 days. Preferably, the length of treatment is at least 5 days. When formulated in a pharmaceutical composition, the compositions of the present invention can be admixed with a pharmaceutically acceptable carrier or excipient. The phrase “pharmaceutically acceptable” refers to molecular entities and compositions that are “generally regarded as safe”, e.g., that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicles with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. Alternatively, the carrier can be a solid dosage from carrier, including but not limited to one or more of a binder (for compressed pills), an encapsulating agent, a flavorant, and a colorant. Suitable pharmaceutical carriers are described in “Remington's Pharmaceutical Sciences” by E. W. Martin. The compositions of the present invention can be formulated into any form known in the art using procedures available to one of skill in the art. The compositions of the present invention may be mixed with other food forms and consumed in solid, semi-solid, suspension or emulsion form. In one embodiment, the composition is formulated into a capsule or tablet using techniques available to one of skill in the art. However, the present compositions may also be formulated in another convenient form, such as an injectable solution or suspension, a spray solution or suspension, a lotion, a gum, a lozenge, a food or snack item. Food, snack, gum or lozenge items can include any ingestible ingredient, including sweeteners, flavorings, oils, starches, proteins, fruits or fruit extracts, vegetables or vegetable extracts, grains, animal fats or proteins. Thus, the present compositions can be formulated into cereals, snack items such as chips, bars, gum drops, chewable candies or slowly dissolving lozenges. The compositions of the present invention can also be administered as dry powder or metered dose of solution by inhalation, or nose-drops and nasal sprays, using appropriate formulations and metered dosing units.

In a specific embodiment, a pharmaceutical composition of the invention comprises: MDSCs in combination with GA or SP600125 alone, or MDSCs in combination with GA and SP600125, and a pharmaceutically acceptable carrier or diluent for intravenous or subcutaneous administration.

In some embodiments antioxidants may be administered in conjunction. Additionally, in some embodiments cells may be administered together with inhibitors of NF-kappa B. Known inhibitors include: Perrilyl alcohol, Protein-bound polysaccharide from basidiomycetes, Rocaglamides (Aglaia derivatives), 15-deoxy-prostaglandin J(2), Lead, Anandamide, Artemisia vestita, Cobrotoxin, Dehydro ascorbic acid (Vitamin C), Herbimycin A, Isorhapontigenin, Manumycin A, Pomegranate fruit extract, Tetrandine (plant alkaloid), Thienopyridine, Acetyl-boswellic acids, 1′-Acetoxychavicol acetate (Languas galanga), Apigenin (plant flavinoid), Cardamomin, Diosgenin, Furonaphthoquinone, Guggulsterone, Falcarindol, Honokiol, Hypoestoxide, Garcinone B, Kahweol, Kava (Piper methysticum) derivatives, mangostin (from Garcinia mangostana), N-acetylcysteine, Nitrosylcobalamin (vitamin B12 analog), Piceatannol, Plumbagin (5-hydroxy-2-methyl-1,4-naphthoquinone), Quercetin, Rosmarinic acid, Semecarpus anacardiu extract, Staurosporine, Sulforaphane and phenylisothiocyanate, Theaflavin (black tea component), Tilianin, Tocotrienol, Wedelolactone, Withanolides, Zerumbone, Silibinin, Betulinic acid, Ursolic acid, Monochloramine and glycine chloramine (NH2Cl), Anethole, Baoganning, Black raspberry extracts (cyanidin 3-O-glucoside, cyanidin 3-O-(2(G)-xylosylrutinoside), cyanidin 3-O-rutinoside), Buddlejasaponin IV, Cacospongionolide B, Calagualine, Carbon monoxide, Cardamonin, Cycloepoxydon; 1-hydroxy-2-hydroxymethyl-3-pent-1-enylbenzene, Decursin, Dexanabinol, Digitoxin, Diterpenes, Docosahexaenoic acid, Extensively oxidized low density lipoprotein (ox-LDL), 4-Hydroxynonenal (HNE), Flavopiridol, [6]-gingerol; casparol, Glossogyne tenuifolia, Phytic acid (inositol hexakisphosphate), Pomegranate fruit extract, Prostaglandin Al, 20(S)-Protopanaxatriol (ginsenoside metabolite), Rengyolone, Rottlerin, Saikosaponin-d, Saline (low Na+ istonic).

All references are incorporated by reference in their entireties.

### Example 1

### Enhancement of JadiCell® Umbilical Cord Mesenchymal Stem Cell Mediated Reduction of Pulmonary Neutrophilic Infiltration by Cancer Stimulated Myeloid Derived Suppressor Cells

Female BALB/c mice of 20-30 weeks of age were inoculated intramuscularly with 500,000 4T1 breast cancer cells. Tumors where allowed to grow for 2 weeks, after which mice were sacrificed and CD133 expressing splenocytes where isolated using Magnetic Activated Cell Sorting according to the manufacturer's instructions (Milteny Biotech). Isolated cells expressed GR-1 and were considered myeloid suppressor cells. JadiCell umbilical cord mesenchymal stem cells where prepared.

Induction of COPD-like pathology was performed by intravenous administration of 200 micrograms of elastase in a volume of 500 microliters of phosphate buffered saline in BALB/c mice.

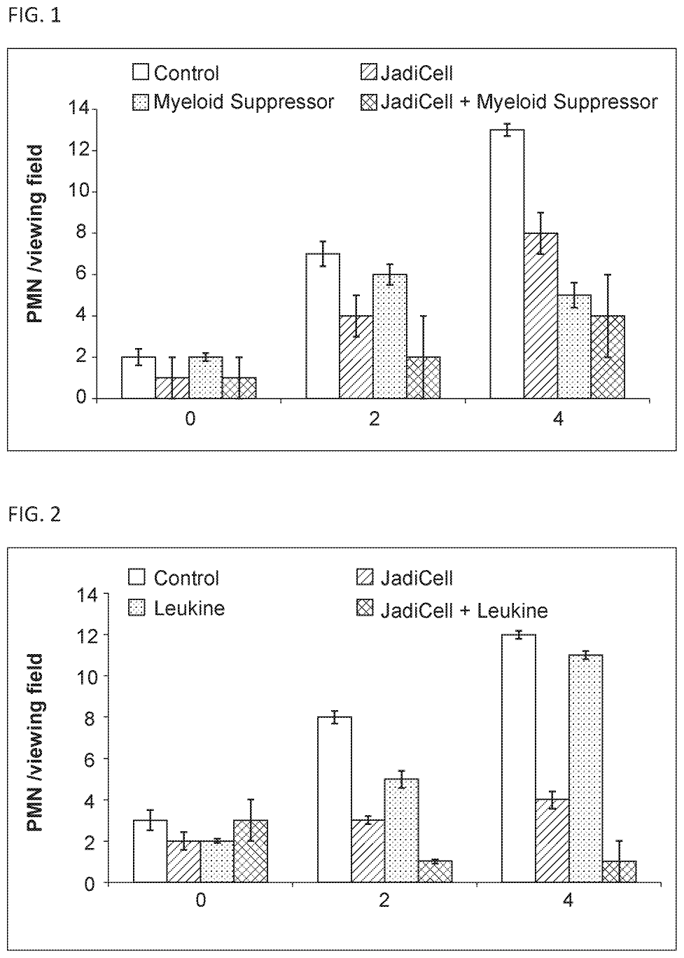

Animals were administered cells concurrently after elastase administration. Quantification of lung polymorphonuclear lymphocyte infiltrations was performed by H & E staining for mice sacrificed at days 0, 2 and 4. Each data point represents 10 mice. A significant reduction in PMN infiltration was observed in mice receiving the combination of JadiCells and Myeloid Derived Suppressor cells. Results are shown in FIG. 1.

### Example 2

### Enhancement of JadiCell Umbilical Cord Mesenchymal Stem Cell Mediated Reduction of Pulmonary Neutrophilic Infiltration by GM-CSF

Female BALB/c mice of 20-30 weeks of age were administered 100 ng per mouse of Leukine brand GM-CSF. JadiCell umbilical cord mesenchymal stem cells where prepared as described¹.

Induction of COPD-like pathology was performed by intravenous administration of 200 micrograms of elastase in a volume of 500 microliters of phosphate buffered saline in BALB/c mice.

Animals were administered cells concurrently after elastase administration. Quantification of lung polymorphonuclear lymphocyte infiltrations was performed by H & E staining for mice sacrificed at days 0, 2 and 4. Each data point represents 10 mice. A significant reduction in PMN infiltration was observed in mice receiving the combination of JadiCells and GM-CSF. Results are shown in FIG. 2.

1. A method of treating Chronic Obstructive Pulmonary Disease (COPD) comprising the steps of: a) identifying a subject suffering from COPD; b) obtaining a mesenchymal stem cell population; c) inducing an increase in endothelial progenitor cell numbers and/or activity in the blood of said patient; and d) administering said mesenchymal stem cell population to the patient suffering from COPD.

2. The method of claim 1, wherein administration of said mesenchymal stem cell population is performed before, concurrently with, or subsequent to increasing endothelial progenitor cell population and/or activity in the blood.

3. The method of claim 1, wherein said mesenchymal stem cell population is either autologous, allogeneic or xenogeneic.

4. The method of claim 1, wherein inducing increase in said endothelial progenitor cell population is induced by administration of exogenous endothelial progenitor cells.

5. The method of claim 4, wherein said exogenous endothelial progenitor cells are derived from a source that is either autologous, allogeneic, or xenogeneic.

6. The method of claim 4, wherein said exogenous endothelial progenitor cells are obtained from adipose tissue.

7. The method of claim 4, wherein said exogenous endothelial progenitor cells are obtained from bone marrow.

8. The method of claim 4, wherein said exogenous endothelial progenitor cells are obtained from umbilical cord tissue.

9. A method of treating COPD comprising administration of: a) either myeloid derived suppressor cells or one or more compounds capable of inducing an increase in number and/or activity of myeloid derived suppressor cells and b) a mesenchymal stem cell population to a subject suffering from COPD.

10. The method of claim 9, wherein said compound capable of increasing number and/or activity of said mesenchymal stem cell is GM-CSF.

11. The method of claim 9, wherein said mesenchymal stem cell is JadiCell.

12. A method of treating COPD comprising administration of: a) GM-CSF and b) a mesenchymal stem cell population to a subject suffering from COPD.
